(19) United States
(12) Patent Application Publication
(10) Pub. No.: US 2011/0028926 A1
(43) Pub. Date: Feb. 3, 2011

(54) Diaper Rash Prevention Apparatus

(76) Inventor: Joseph E. Davis, Morgan Hill, CA (US)

Correspondence Address: JOSHUA D. ISENBERG;JDI PATENT, 809 CORPORATE WAY, FREMONT, CA, 94539, US

(21) Appl. No.: 12/904,911

(22) Filed: Oct. 14, 2010

Related U.S. Application Data
(63) Continuation-in-part of application No. 11/384,145, filed on Mar. 16, 2006.
(60) Provisional application No. 61/252,566, filed on Oct. 16, 2009.

(51) Int. Cl. A61F 13/15; A61B 19/02

(52) U.S. Cl. 604/359; 604/385.101; 206/438

(57) ABSTRACT

A diaper rash prevention apparatus includes a single layer enclosure with a flangeless aperture adapted for being placed in direct contact with the skin surrounding the anus for the purpose of directing fecal matter into the single layer enclosure. An adhesive may be disposed about the flangeless aperture to maintain the aperture in direct contact with the skin surrounding the anus. A cover may be releasably coupled over at least one of the flangeless aperture and the adhesive. An applicator may be used to assist in the installation of the single layer enclosure.

### CLAIM OF PRIORITY

This application is a continuation in part of and claims the priority benefit of commonly-owned, co-pending U.S. patent application Ser. No. 11/384,145, to Joseph E. Davis, filed Mar. 16, 2006, and entitled “DIAPER RASH PREVENSION APPARATUS” the entire disclosures of which are incorporated herein by reference.

This application claims the priority benefit of commonly-owned co-pending U.S. Provisional Patent Application No. 61/252,566, to Joseph E. Davis, filed Oct. 16, 2009, and entitled “DIAPER RASH PREVENSION APPARATUS” the entire disclosures of which are incorporated herein by reference.

### FIELD AND BACKGROUND OF THE INVENTION

Embodiments of the present invention relate to sanitary apparatuses, and more particularly, to sanitary apparatuses adapted for managing fecal matter.

### SUMMARY

A diaper rash prevention apparatus may comprise a single layer enclosure with a flangeless aperture adapted for being placed in direct contact with the skin surrounding the anus for the purpose of directing fecal matter into the single layer enclosure. An adhesive may be disposed about the flangeless aperture to maintain the aperture in direct contact with the skin surrounding the anus. A cover may be releasably coupled over at least one of the flangeless aperture and the adhesive. The single layer enclosure may be constructed utilizing an air-impermeable or water-impermeable material.

A sleeve or pocket for receiving an applicator may be attached to the single layer enclosure. The sleeve may be attached to the single layer enclosure along at least two edges of the single layer enclosure such that the single layer enclosure is between the sleeve and the wearer. The side of the single layer enclosure that contains the aperture faces the wearer.

In some versions, the adhesive may include a transfer adhesive or a double-sided adhesive.

In some versions, an applicator may be placed inside the sleeve or pocket to facilitate installation of the single layer enclosure on the wearer.

In some versions, there is no sleeve or pocket and an applicator is used to facilitate installation of the single layer enclosure on the wearer.

In some versions, the sleeve or pocket is replaced by tabs that are attached to the single layer enclosure. The tabs perform the same function as the sleeve or pocket. An applicator is used to facilitate installation of the single layer enclosure on the wearer by grasping the tabs and the applicator simultaneously.

In some versions, at least one area of adhesive is placed on the sheet of the single layer enclosure facing away from the user and an applicator is attached to the adhesive to hold the diaper rash prevention device in position to facilitate installation of the diaper rash prevention device.

In some versions, the applicator may be thin, inflexible, and rectangular with rounded edges and at least two rounded corners. In some versions the applicator may be thin, inflexible, and any shape so long as one edge is straight and extends approximately the width of the adhesive area on the single layer enclosure.

In some versions the single layer enclosure may comprise a polyethylene material or a biodegradable plastic material or any biodegradable water-impermeable material with a thickness of less than 50 microns.

In some versions the adhesive is comprised of a material such as an acrylic that although is not biodegradable itself, it decomposes or breaks down and becomes inert with the environment if the enclosure material that it is attached to is biodegradable and in the case of a double-sided adhesive the carrier material between the two layers of adhesive is biodegradable.

A device and a method for facilitating the installation of a diaper rash prevention apparatus are described.

### BRIEF DESCRIPTION OF THE DRAWINGS

FIG. 1A is a schematic diagram illustrating a top view of an apparatus for prevention of diaper rash according to an embodiment of the present invention.

FIG. 1B is a cross-section view of the apparatus of FIG. 1A taken along dotted line B-B.

FIG. 2 is a side view of the apparatus 100 folded in half to facilitate installation according to an embodiment of the present invention.

FIG. 3A is a side view of an applicator.

FIG. 3B is an edge view of an applicator.

FIG. 4 is a side view illustrating an applicator inside Diaper Rash Prevention Device Sleeve and held by Installer.

FIG. 5 is a top view photo of a Diaper Rash Prevention Device of FIG. 1.

FIG. 6 shows a photograph (white area only) of a side view of another embodiment of an Applicator.

FIG. 7 is a flow diagram illustrating a procedure for installing a Diaper Rash Prevention Device on an intended wearer.

FIG. 8 is a photograph showing a Diaper Rash Prevention Device Being Installed onto the perianal area of an intended wearer.

FIG. 9 is a photograph showing a Diaper Rash Prevention Device Installed and Ready for Diaper Closure.

FIG. 10 is a photograph of a side view of a folded Sleeveless Diaper Rash Prevention Device according to another embodiment of the present invention.

FIG. 11 is a photograph of a side view of a folded Sleeveless Diaper Rash Prevention Device with Applicator inserted and both held by installer.

### DETAILED DESCRIPTION

A diaper rash prevention device is described in US Patent Application Publication number 20070215593 A1, the entire contents of which are incorporated herein by reference. US 2007/0215593 A1 describes an apparatus that includes an enclosure with an aperture which is adapted for being placed in communication with an anus for the purpose of directing fecal matter into the enclosure.

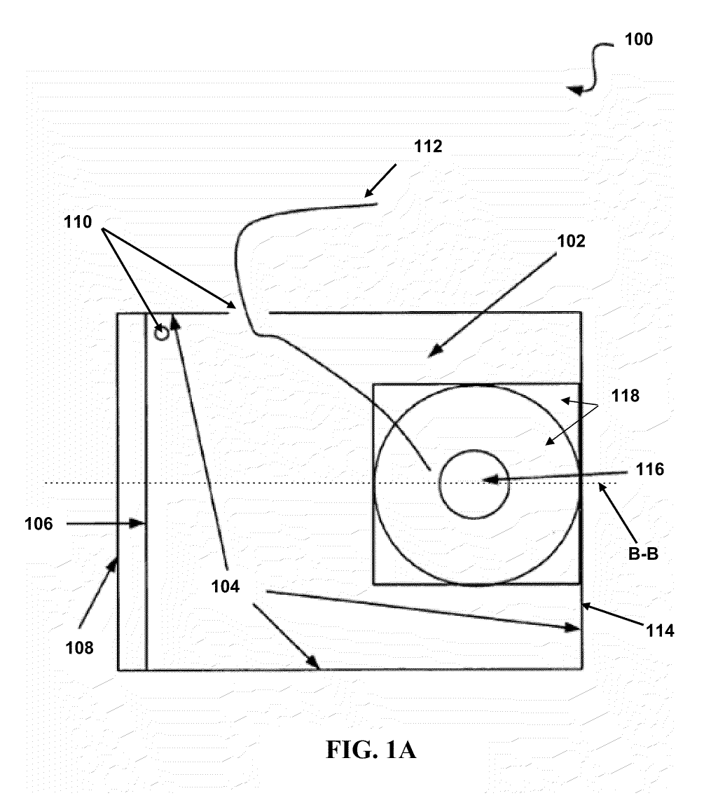

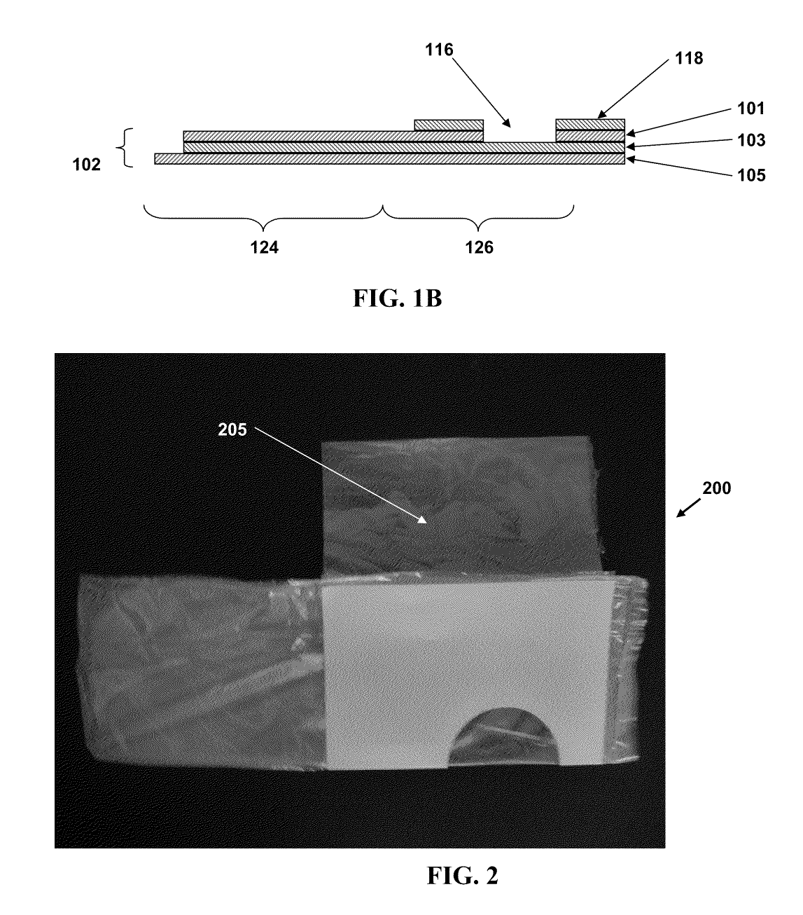

In one embodiment, the enclosure may include a polyethylene material or a biodegradable plastic material with a thickness of less than 50 microns. Additionally, in various embodiments, the thickness of the sheet comprising the walls of the enclosure 100 shown in FIG. 1A may be approximately 0.6 mils (i.e. 15 microns), but may also be substantially thinner or thicker (e.g. 10, 20, 25, 30 microns, etc.) depending on the age, weight, or gender of the intended wearer while still remaining sufficiently flexible, comfortable, and waterproof. Specifically, as the thickness of the enclosure 100 exceeds 1.25 mils (i.e. approximately 31 microns), it may become less flexible such that it does not always conform well to the perianal area of the intended wearer, thereby causing the enclosure 100 to perform less reliably. Conversely, as the thickness of the enclosure 100 becomes thinner (below 31 microns), it becomes more flexible which enables it to conform better to the perianal area of the intended wearer. In addition, as the thickness of the enclosure 100 is reduced below 31 microns, the material becomes more breathable which is beneficial to the skin of the intended wearer. A further advantage of reducing the thickness of the enclosure 100 is that the time for biodegradation (if the enclosure 100 is comprised of a biodegradable material) decreases as the thickness of the material decreases.

In another embodiment, the enclosure may include an air hole formed therein for remaining open to allow air to escape therefrom upon the receipt of the fecal matter into the enclosure. In still yet another embodiment, the aperture may be circular. FIG. 1A illustrates a top view of an apparatus 100 for prevention of diaper rash. The apparatus 100 includes an enclosure that prevents diaper rash. For clarity, a cross-section along dotted line B-B of FIG. 1A is shown in FIG. 1B. Specifically, FIGS. 1A-1B show an apparatus 100 that includes three sheets 102, where a top sheet layer 101 facing the wearer and a middle sheet layer 103 are sealed to each other on all four edges 104 and 106 while a third sheet layer 105 is sealed to the other two sheet layers 101, 103 along three of the edges 104 with the fourth edge 108 left unsealed. Although it is shown in FIG. 1A that the unsealed fourth edge 108 of the third sheet layer extends to the left beyond the sealed edge 106 of the wearer facing layer sheet and the middle layer sheet, it may also be coincident with edge 106 or to the right of edge 106 without affecting its purpose and may be chosen on the basis of convenience or ease of manufacture. The third sheet layer described in FIG. 1B can be used to assist in the installation of the Diaper Rash Prevention device. The third sheet layer acts as a three-sided pocket that allows the hand of the installer to be placed between the third sheet layer and the middle sheet layer through the unsealed fourth edge 108.

The top layer 101 may include an adhesive portion 118, e.g., a double-sided adhesive or transfer adhesive surrounding an aperture 116. The top side of the adhesive 118 is in direct contact with the skin at the perianal region of the intended wearer. The bottom side of adhesive 118 is in direct contact with the top sheet layer 101 of the apparatus 100. In some versions the transfer adhesive 118 is comprised of a material such as an acrylic adhesive that although is not biodegradable itself, it decomposes or breaks down and becomes inert with the environment if the top sheet layer 101 that it is attached to is biodegradable. If the adhesive 118 is a double-sided adhesive comprised of a material such as an acrylic adhesive and it is desired that it decompose or break down and become inert with the environment, then both the top sheet layer 101 and the carrier material between the two layers of adhesive may be made of a biodegradable material.

Once the enclosure 100 is placed on the intended wearer, the only opening remaining in the enclosure 100 that is exposed to the outside environment is at least one small hole 110, such as a pinhole, that is approximately 40-50 mils in diameter (i.e. 1-1.25 mm in diameter). The small hole 110 allows for the escape of gas or air trapped in the enclosure 100. This hole 110 can be placed as far from the aperture 116 over the anal opening as possible to minimize any leaking of solid waste through it. For example, the hole 110 may be located on an end of the enclosure 100 that is opposite to the end of the enclosure 100 proximal to the aperture 116. The at least one hole 110 may be placed along an edge of the enclosure 100 or on the face of one of the sheets comprising the enclosure 100. Although leakage may rarely occur through the hole 110, it is minimal in nature. However, the hole 110 prevents the enclosure 100 from being placed under excessive pressure from trapped air or gas while the wearer is in a sitting position which may cause the enclosure 100 to burst.

Additional optional enhancements may also be provided in the context of the enclosure 100 of FIG. 1A, including: 1) medication, chemicals and/or perfumes placed inside the enclosure 100 to chemically react with the solid waste to at least further reduce or eliminate odor, 2) a bio-degradable enclosure 100 such as those produced from corn sugars by NatureWorks, a subsidiary of Cargill, that would be environmentally disposable and/or could be safely placed into and flushed in a toilet for disposal, 3) a string or strip 112 whose one end is inside the enclosure 100 and whose other end exits through a small opening in the enclosure 100 (e.g. the air hole 110) that changes color or appearance to indicate that the enclosure has been filled with solid waste, such as a strip of litmus material

While the method described above for installing the apparatus 100 is acceptable, it is desirable to make the apparatus 100 as easy and convenient to install as possible. An alternative embodiment of the Diaper Rash Prevention device 100 utilizes the third sheet layer 105 as a two-sided sleeve rather than a three-sided pocket. This is accomplished by leaving edges 108 and 114 of the third sheet layer unsealed and sealing two parallel edges 104 (but not sealing the portion of edge 104 that is coincident with edge 114). The second edge 114 left unsealed is the edge opposite the fourth edge 108 already left unsealed. This alternate embodiment of the diaper rash prevention device 100 can be folded in half along the dotted line B-B of FIG. 1A prior to installing it on the intended wearer. Doing so allows the apparatus 100 to conform better to the shape of the perianal region of the intended wearer. A side view of one embodiment of the apparatus 100 folded in half in this manner is shown in FIG. 2.

Note that when the diaper rash prevention device is folded in this manner, it is possible to extend the third sheet layer (sleeve) 105 upward (as shown in FIG. 2) away from the top sheet layer 101 and middle sheet layer 103 forming the enclosure. Note also that the third sheet layer 105 in FIG. 2 is not as wide as the third sheet layer 105 shown in FIGS. 1A-1B. Although it is shown in FIG. 1B that the unsealed fourth edge 108 of the third sheet layer extends to the left beyond the sealed edge 106 of the top sheet 101 and the middle layer sheet 103, it may also be coincident with sealed edge 106 or to the right of the sealed edge 106 without affecting its purpose and may be chosen on the basis of convenience or ease of manufacture.

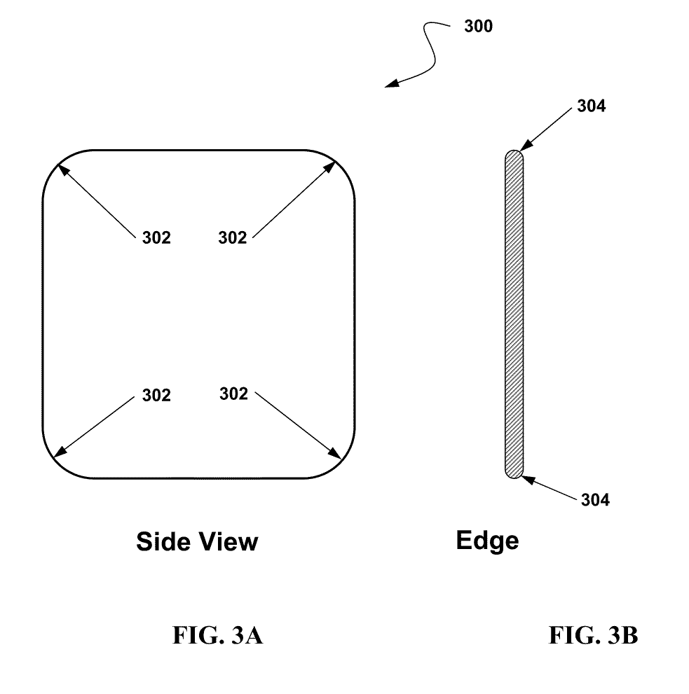

When the hand of the installer is placed inside the sleeve formed by the third sheet layer 105 and the middle sheet layer 103, it may cause the folded Diaper Rash Prevention device 200 to become unfolded thereby making it difficult to maintain it in the narrow folded manner as shown in FIG. 2. Hence, it is helpful to have a thin rectangular shaped block, called an Applicator that can be inserted into the extended sleeve 205 formed by the third sheet layer 105 and the middle sheet layer 103. One embodiment of such an Applicator 300 is shown in FIGS. 3A-3B. The dimensions of the Applicator may be nominally 3.50 inches by 3.5 inches and about 3/16 inch thick but could be any set of dimensions that allows the Applicator 300 to fit conveniently inside the extended sleeve 205 formed between the third sheet layer 105 and the middle sheet layer 103. In addition, the applicator need not be rectangular. It can be any shape so long as there is at least one straight edge that is approximately as wide as the width of the adhesive area 118. The width of the adhesive area 118 may be defined as from the leftmost edge of the adhesive area 118 to the rightmost edge of the adhesive area 118 as shown in FIG. 1B and denoted as the width 126. The applicator can be made out of any suitable firm material such as Styrofoam, plastic, metal, wood, glass, etc. All edges 304 may be rounded and at least two of the corners 302 may be rounded to eliminate the possibility of any discomfort to the intended wearer during installation of the Diaper Rash Prevention device 100.

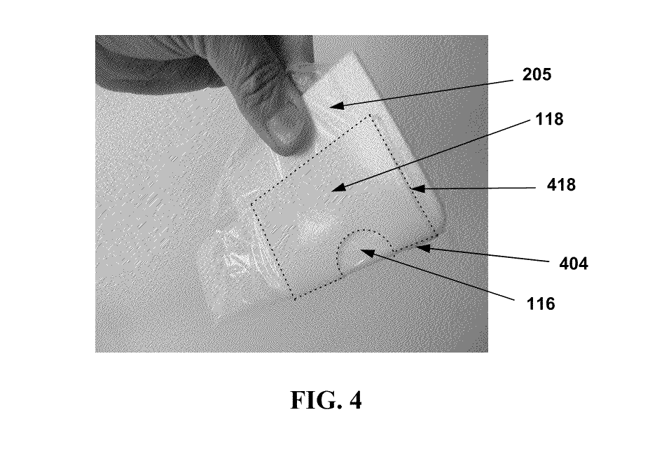

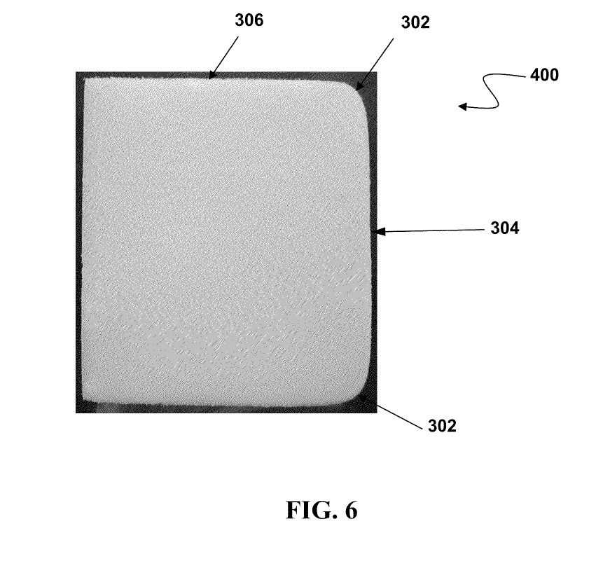

If the applicator 300 is constructed in this manner, it will fit inside the extended sleeve 205 so that the folded Diaper Rash Prevention device 200 remains folded and narrow as desired. FIG. 4 shows another embodiment of an Applicator 400 (shown in FIG. 6) inserted into the extended sleeve 205 of the folded Diaper Rash Prevention device 200. It may be held by the installer by grasping the extended third sheet layer sleeve 205 (with the Applicator 400 inside) as shown in FIG. 4.

An applicator may be reusable and may be provided in a package containing one or more diaper rash prevention devices or may be provided separately. By way of example, and not by way of limitation, an applicator may be included in each box of 50 or so diaper rash prevention devices.

Note that the extended third sheet layer sleeve 205 with Applicator 400 inside is held by the installer with a thumb and forefinger so that the fingers do not come into contact with a release liner 418 covering the adhesive 118 (the release liner is approximately denoted by the area 418 within the dotted lines of FIG. 4) surrounding the aperture 116 of the folded diaper rash prevention device 200. This prevents interfering with the removal of the adhesive release liner 418 and also prevents having the fingers of the installer come into contact with the adhesive 118 once the release liner 418 is removed.

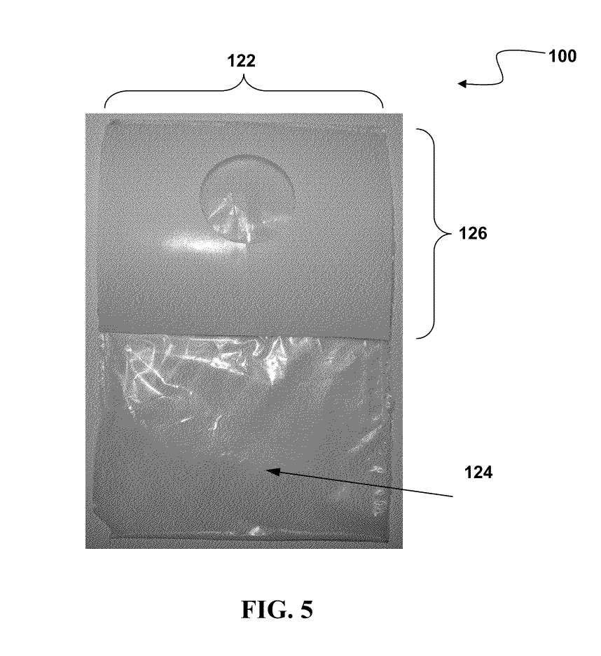

FIG. 5 is a photograph of an unfolded top view of an embodiment of the Diaper Rash Prevention device similar to the embodiment 100 shown in FIG. 1A. As may be seen in FIG. 5, the release liner 418 and adhesive 118 may extend across the full length 122 of the top sheet layer 101 that faces the wearer. FIG. 6 shows a photograph (white area only) of a side view of another embodiment of the Applicator 400. It differs from the embodiment of the Applicator 300 shown in FIG. 3 in that it has only two rounded corners and the width and length are not the same.

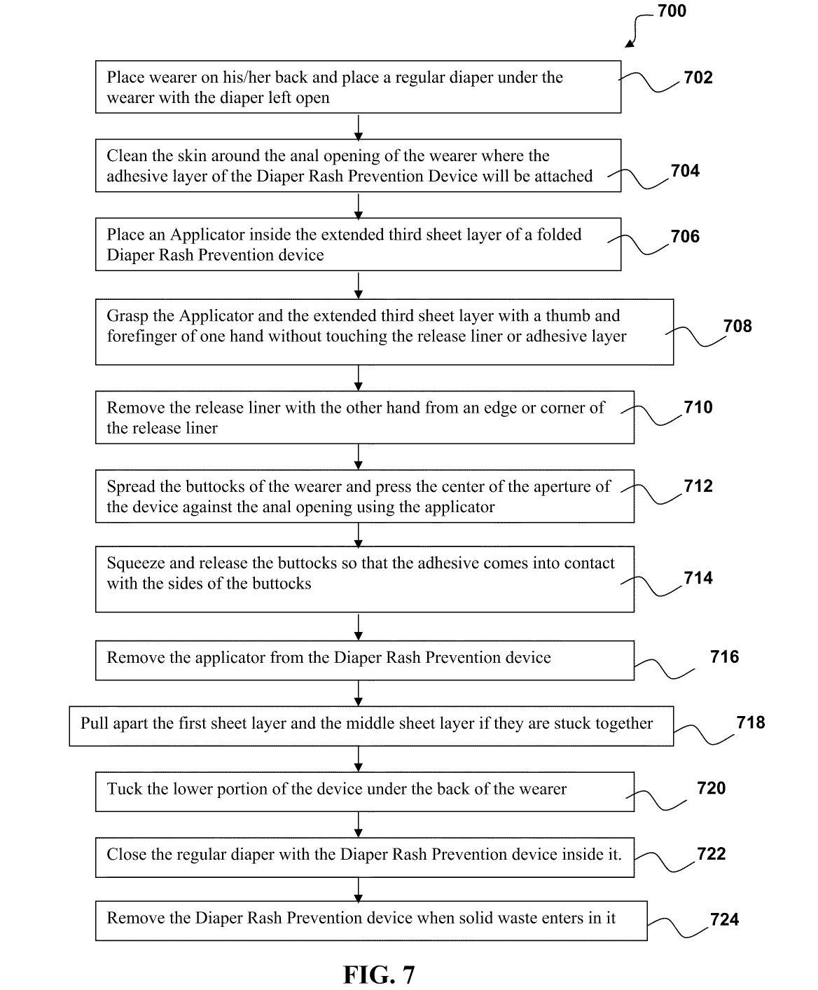

An example of a procedure 700 for installing the Diaper Rash Prevention device 100 on an intended wearer is shown in the flow diagram of FIG. 7. The intended wearer may be placed on his/her back. A regular diaper can be placed under the wearer but should be left open as indicated at 702. It is desirable for the skin around the anal opening where the adhesive 118 will be attached to be clean and dry. The skin may be cleaned with soap and water and/or rubbing alcohol as indicated at 704. No-Sting Liquid Bandage Spray (such as Nexcare from Minnesota Mining and Manufacturing Company of Saint Paul, Minn.) may also be sprayed around the anal opening where the adhesive 118 will be attached to the skin and spread evenly with a finger or any spreading implement until the spray becomes sticky to improve the adherence of the adhesive 118 to the skin. The Diaper Rash Prevention device 100 may be folded in half along the line B-B of FIG. 1A as shown in FIG. 2. The Applicator 400 is then placed inside the extended third sheet layer sleeve 105 of the folded Diaper Rash Prevention device 100 (as shown in FIG. 4) as indicated at 706. As shown above in FIG. 6, the Applicator 400 only has two rounded corners 302 along edge 304. Hence, the edge 304 of the Applicator 400 may be located and pressed snugly along the bottom inside edge 404 of the folded Diaper Rash Prevention device 100 as shown in FIG. 4. One-half of the adhesive area 118 and aperture 116 may be on one side of the applicator 400 and the other half on the other side of the applicator 400 as shown in FIG. 4. The edge 304 may be longer than the width of the adhesive 126 in FIG. 5 so that the short edge 306 of the applicator 400 extends, e.g., about IA″ inch beyond the edge 418 of the adhesive covered area 118. This insures that the flat portion of the edge 304 is coincident with the full width of the adhesive coated area 118 to maximize contact of the adhesive 118 with the skin when installed on the wearer.

After the Applicator 400 has been placed inside the extended third sheet layer sleeve 105, as indicated at 708, the installer may grasp both the Applicator 400 and the extended third sheet layer sleeve 105 with a thumb and forefinger of one hand as shown in FIG. 4 being careful not to grasp any of the area 118 where the release liner 418 and adhesive 118 are located. This avoids interfering with the removal of the release liner 418 and avoids coming into contact with any portion of the adhesive 118 after the release liner 418 has been removed.

The release liner 418 may then be removed from the exterior facing side of the adhesive 118 with the hand not grasping the Diaper Rash Prevention device 100 as indicated at 710. The removal of the release liner 418 may be facilitated if there is a small portion of an edge or a corner of the release liner 418 that does not have adhesive 118 coated on it. This would provide an edge or corner of release liner 418 that can be grasped easily to initiate the removal of the adhesive 118.

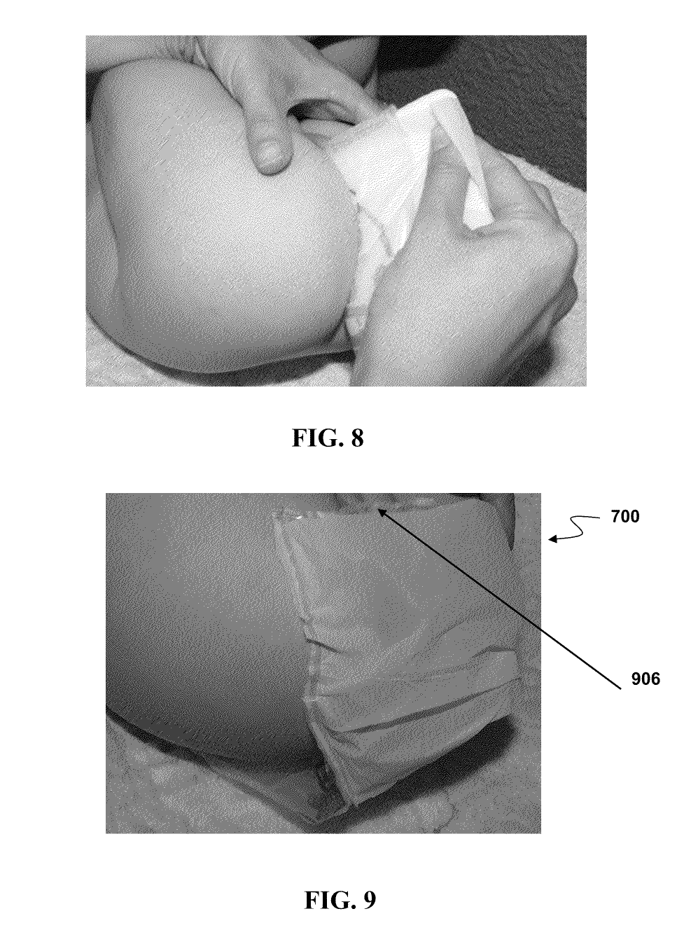

The buttocks of the intended wearer are then spread apart while pressing his/her legs against his/her stomach as shown in FIG. 8. The center of the aperture 116 may then be pressed firmly against the anal opening using the applicator 400 as indicated at 712. The area containing adhesive 118 may also be pressed firmly against the skin surrounding the anal opening above and below the anal opening with the applicator 400.

The buttocks may then be released so that the adhesive 118 comes into contact with the sides of the buttocks as shown in FIG. 8 as indicated at 714. The buttocks may be squeezed together to facilitate this contact. It is important that a uniform adhesive seal be formed about the anal opening. Any portion of the adhesive 118 that is still not in firm contact with the skin may be pressed against the skin with the hand of the installer. This adhesive 118 seal prevents solid waste and urine from coming into contact with the skin or each other (the two main causes of diaper rash) and ensures that a bowel movement is fully captured by the Diaper Rash Prevention device 100.

The applicator 400 may then be removed from the Diaper Rash Prevention device 100 by withdrawing it through one of the open ends of the sleeve as indicated at 716.

The installation process may cause the first sheet layer 101 and the middle sheet layer 105 of the Diaper Rash Prevention device 100 to get stuck to one another. The two sheets may be pulled apart taking care not to tear the sealed edges 104 and 106 or to cause the adhesive 118 to become detached from the skin of the wearer as indicated at 718.

The lower portion 124 of the Diaper Rash Prevention device 100 may be tucked under the back of the wearer as indicated at 720. The fully installed Diaper Rash Prevention device 100 is shown in FIG. 9.

A regular diaper can now be closed with the diaper rash prevention device 100 in place inside it as indicated at 722.

Once solid waste enters the Diaper Rash Prevention device 100, it should be removed along with the solid waste contents as indicated at 724. This is accomplished by laying the wearer on his/her back and opening the regular outer diaper. The diaper rash prevention device 100 may then be peeled from the wearer by pulling top edge 906 shown in FIG. 9 downward. If removing the diaper rash prevention device 100 becomes uncomfortable for the wearer, baby oil (such as is commonly available from Johnson & Johnson) may be applied along the seam where the adhesive 118 is being removed from the skin. The baby oil may be rubbed into the skin and the adhesive 118 simultaneously as the adhesive 118 is being removed. Once removed, the diaper rash prevention device 100 with contents may be placed in a toilet or in a container with other diapers or buried in a compost pile. Any small amount of remaining solid waste may be cleaned from the wearer's skin. If the regular diaper is still dry it may continue to be worn.

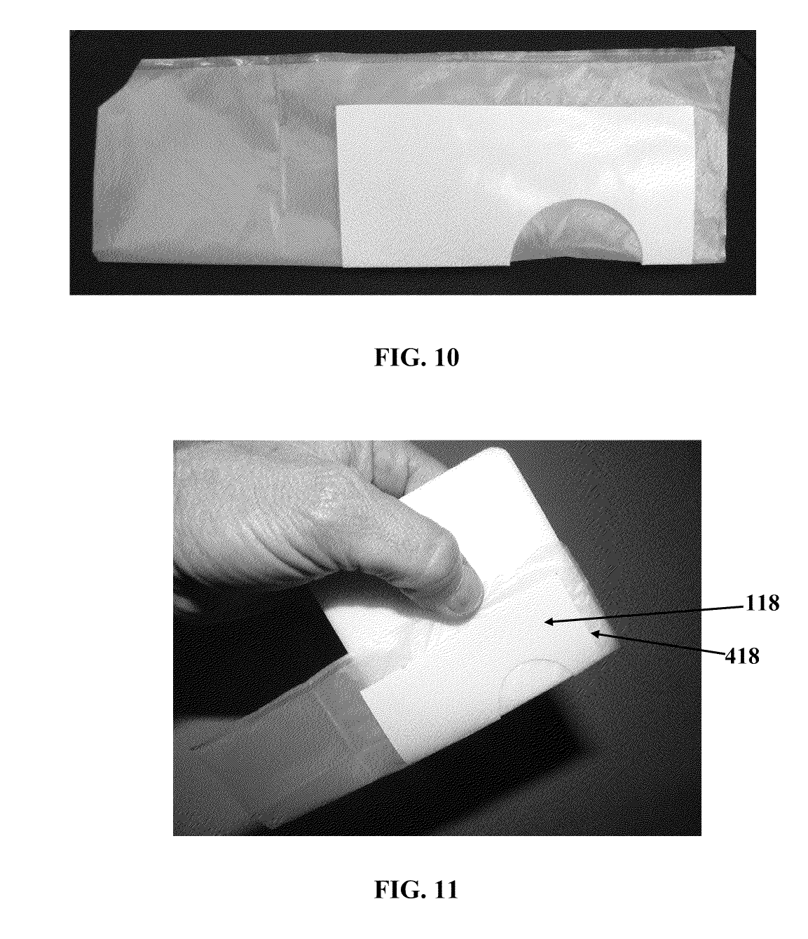

In another embodiment of the present invention, there is no third sheet layer 105 and therefore no sleeve or pocket. However, the single layer enclosure can still receive the Applicator 300 to facilitate the installation of the Diaper Rash Prevention device on the intended wearer. In this embodiment, the Diaper Rash Prevention device is still folded as shown in FIG. 2 but there is no third sheet layer 105 that forms a pocket or sleeve. The folded version of this embodiment is shown in FIG. 10.

In this embodiment, the applicator 300 is still inserted inside the folded diaper rash prevention device 100 as shown in FIG. 11; however, there is no extended sleeve 205 to hold onto. As seen in FIG. 11, the adhesive area 118 and release liner 418 do not extend across the full length 122 of the first sheet layer 101. Therefore, the installer must grasp the Applicator 300 as well as an area of the first sheet layer 101 that does not have the release liner 418 or adhesive 118. The Diaper Rash Prevention device held in this manner is shown in FIG. 11.

In another embodiment, the third sheet layer 105 is in place but not used. That is, the adhesive 118 and release liner 418 do not extend across the full length 122 of the first sheet layer 101. The applicator 300 and Diaper Rash Prevention device 100 are grasped as shown in FIG. 11. The third sheet layer 105 is in place but not extended. Hence, the applicator 300 presses against the exterior of the third sheet layer 105 rather than placed between the third sheet layer 105 and the middle sheet layer 103.

In another embodiment, the third sheet layer 105 can be cut into two separate sheets (for example, along the top of the extended sleeve 205 from left to right). Each separate sheet is attached to the single layer enclosure along opposite edges. The installer still grasps the two separate sheets and the applicator as shown in FIG. 4 except that the extended sleeve 205 includes two separate sheets not attached to each other but still attached to the single layer enclosure along at least two edges.

In another embodiment, there is no third sheet 105 and therefore no pocket or sleeve. However, an adhesive is placed on at least one part of the exterior surface of the middle sheet layer 103 and may be covered by a releasable liner. Once the releasable liner is removed, the adhesive may be attached to the applicator to hold the diaper rash prevention device to the applicator to facilitate installation of the diaper rash prevention device.

Although the present invention has been described in considerable detail with reference to certain preferred versions thereof, other versions are possible. For example, the applicator may be provided with the diaper rash prevention device or provided separately. Furthermore, the enclosure, adhesive, and applicator may be made with other materials. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents incorporated herein by reference.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

Any feature, whether preferred or not may be combined with any other feature, whether preferred or not.

Any element in a claim that does not explicitly state “means for” performing a specified function, is not to be interpreted as a “means” or “step” clause as specified in 35 USC §112, ¶ 6. In particular, the use of “step of” in the claims herein is not intended to invoke the provisions of 35 USC §112, ¶ 6.

What is claimed is:

1. An apparatus, comprising:
a single layer enclosure with a flangeless aperture adapted for being placed in direct contact with the skin surrounding the anus for the purpose of directing fecal matter into the single layer enclosure;
wherein the single layer enclosure comprises a polyethylene material or a biodegradable plastic material with a thickness of less than 50 microns;
wherein an adhesive is formed about the flangeless aperture for maintaining the flangeless aperture in direct contact with the skin surrounding the anus;
wherein a cover is releasably coupled over at least one of the flangeless aperture and the adhesive;
wherein the single layer enclosure is constructed utilizing an air-impermeable or water-impermeable material;
wherein the adhesive includes a transfer adhesive;
wherein the single layer enclosure is configured to receive an applicator to assist in the installation of the single layer enclosure;
wherein the applicator is thin, inflexible, and has at least one straight edge that extends approximately the width of an area of the single layer enclosure containing the adhesive.

2. The apparatus of claim 1, wherein the single layer enclosure comprises a pair of substantially planar sheets each with edges that are integrally coupled.

3. The apparatus of claim 1, wherein a substance is included inside the single layer enclosure, the substance selected from the group consisting of a medication, an odor neutralizing chemical, and a perfume.

4. The apparatus of claim 1, wherein the adhesive includes a double-sided adhesive.

5. The apparatus of claim 1, wherein at least one air hole is formed in the single layer enclosure for remaining open to allow air to enter therein to prevent the walls of the single layer enclosure from sticking together or escape therefrom upon receipt of the fecal matter into the single layer enclosure

6. The apparatus of claim 5, wherein a piece of material passes through the air hole in the single layer enclosure, wherein the piece of material is configured to indicate whether the fecal matter has been received into the single layer enclosure.

7. The apparatus of claim 1, wherein a liquid adhesive is applied to the skin surrounding the anus of the wearer to enhance the adhesion of the adhesive.

8. The apparatus of claim 1, wherein the cover releasably coupled over at least one of the flangeless aperture and the adhesive has a small portion of an edge or a corner of the cover that does not have adhesive adhered to it so as to facilitate the initiation of removal of the cover from the adhesive.

9. The apparatus of claim 1 wherein the transfer adhesive comprises a material that may not be biodegradable by itself but decomposes or breaks down to become inert with the environment if the enclosure material it is attached to is biodegradable.

10. The apparatus of claim 4 wherein the double-sided adhesive comprises a material that may not be biodegradable by itself but decomposes or breaks down to become inert with the environment if the enclosure material it is attached to is biodegradable and the carrier material between the two layers of adhesive is biodegradable.

11. The apparatus of claim 1 wherein the single layer enclosure includes a sleeve for receiving an applicator to facilitate installation of the single layer enclosure on the wearer.

12. The apparatus of claim 11 wherein the sleeve is attached to the single layer enclosure along at least two edges of the single layer enclosure such that the single layer enclosure is between the sleeve and the wearer and the side of the single layer enclosure containing the aperture faces the wearer.

13. The apparatus of claim 1 wherein the single layer enclosure includes two separate sheets for use in conjunction with an applicator to facilitate installation of the single layer enclosure on the wearer.

14. The apparatus of claim 13 wherein each separate sheet is attached to the single layer enclosure along opposing edges of the single layer enclosure such that the single layer enclosure is between the separate sheets and the wearer and the side of the single layer enclosure containing the aperture faces the wearer.

15. The apparatus of claim 1 wherein at least one area of adhesive is placed on the side of the single layer enclosure facing away from the wearer for attaching to an applicator to facilitate installation of the single layer enclosure on the wearer.

16. The apparatus of claim 15 wherein the area of adhesive is covered by a releasable liner.

17. A diaper rash prevention kit, comprising:
one or more diaper rash prevention devices; and
one or more applicators, wherein the one or more diaper rash prevention devices and the one or more applicators are packaged together;
wherein each of the one or more diaper rash prevention devices comprises a single layer enclosure with a flangeless aperture adapted for being placed in direct contact with the skin surrounding the anus for the purpose of directing fecal matter into the single layer enclosure, wherein the single layer enclosure comprises a polyethylene material or a biodegradable plastic material with a thickness of less than 50 microns; wherein an adhesive is formed about the flangeless aperture for maintaining the flangeless aperture in direct contact with the skin surrounding the anus, wherein a cover is releasably coupled over at least one of the flangeless aperture and the adhesive, wherein the single layer enclosure is constructed utilizing an air-impermeable or water-impermeable material, wherein the adhesive includes a transfer adhesive, wherein the single layer enclosure is configured to receive an applicator to assist in the installation of the single layer enclosure;
wherein each of the one or more applicators is thin, inflexible, and has at least one straight edge that extends approximately the width of an area of the single layer enclosure containing the adhesive.
